(19) United States
(12) Patent Application Publication
(10) Pub. No.: US 2015/0073131 A1
(43) Pub. Date: Mar. 12, 2015

(54) METHOD FOR PRODUCING BIOBASED CHEMICALS FROM PLANT LIGNIN

(71) Applicant: Vertichem Corporation, Cambridge (CA)

(72) Inventors: John R. Peterson, Chardon, OH (US); Christopher M. Yost, Ayr (CA); Jian Wu, Guelph (CA)

(21) Appl. No.: 14/384,005

(22) PCT Filed: Mar. 11, 2013
(86) PCT No.: PCT/US2013/030104

§ 371 Date: Sep. 9, 2014

Related U.S. Application Data
(60) Provisional application No. 61/608,936, filed on Mar. 9, 2012.

(51) Int. Cl. C08H 7/00; C07C 45/00; C07C 1/22; C07C 67/00; C07C 41/01; C10G 1/04; C07C 37/00; C07C 51/00

(52) U.S. Cl. CPC C08H 6/00; C07C 37/00; C07C 45/00; C07C 51/00; C07C 67/00; C07C 41/01; C10G 1/045; C07C 1/22; C07C 2523/22; C07C 2523/28; C07C 2523/44; C07C 2523/46; C07C 2523/75; C07C 2523/755
USPC 530/500; 568/716; 562/405; 568/426; 568/309; 568/675; 562/475; 560/103; 568/650; 562/476; 585/240; 585/242; 585/321; 585/320; 585/319; 585/469

(57) ABSTRACT

The present invention is directed generally to a method of production of value-added, biobased chemicals from lignin sources, including waste lignin. A method of producing biobased aromatic chemicals, biobased aromatic fuels, and/or lignin residues from lignin is also described herein.

This application claims priority to U.S. Ser. No. 61/608,936 entitled A METHOD FOR PRODUCING BIOBASED CHEMICALS FROM PLANT LIGNIN filed on Mar. 9, 2012 and PCT Application No. PCT/US2013/030104 entitled A METHOD FOR PRODUCING BIOBASED CHEMICALS FROM PLANT LIGNIN filed on Mar. 11, 2013 which are incorporated by reference.

### I. BACKGROUND OF THE INVENTION

A. Field of Invention

The present invention is directed generally to a method of production of value-added, biobased chemicals from lignin sources, including waste lignin. A method of producing biobased aromatic chemicals, biobased aromatic fuels, and/or lignin residues from lignin is also described herein.

B. Description of the Related Art

The world currently faces depletion of fossil fuels while demands for these fuels are ever increasing. Petrochemicals provide an energy source and a component of the majority of raw materials used in many industries. In fact, approximately 95% of all chemicals manufactured today are derived from petroleum. However, this heavy reliance upon fossil fuels is creating harm to the environment. The burning of these fossil fuels has led to the pollution of air, water and land, as well as global warming and climate changes. Through the use of fossil fuels, the environment has been harmed, perhaps irreparably, in an effort to meet the nearly insatiable demand for energy and manufactured products. Fossil fuels are a finite natural resource. With the depletion of readily available oil reserves across the globe, the supply chain has shifted to more complex and environmentally risky production technologies. A reduction and conservation of fossil fuels is clearly needed. Some alternatives to fossil fuels, like solar power, wind power, geothermal power, hydropower, and nuclear power, are used to a degree. However, a more efficient use of renewable resources is always being sought.

As a stable and independent alternative to fossil fuels, biomass can be a potentially inexhaustible, domestic, natural resource for the production of energy, transportation fuels, and chemicals. The advantage in use of biomass for such purposes is magnified during an oil crisis, a surge in oil prices, or political unrest within oil producing regions of the world. Biomass includes plant and wood biomass, including agricultural biomass. Biomass can be employed as a sustainable source of energy and is a valuable alternative to fossil fuels. More specifically, the biorefining of biomass into derivative products typically produced from petroleum can help to stop the depletion of petroleum, or at least reduce the current demand and dependence. Biomass can become a key resource for chemical production in much of the world. Biomass, unlike petroleum, is renewable. Biomass can provide sustainable substitutes for petrochemically derived feedstocks used in existing markets.

Biomass is made up primarily of cellulose, hemicellulose, and lignin. These components, if economically separated from one another, can provide vital sources of chemicals normally derived from petrochemicals. The use of biomass can also be beneficial with agricultural and/or woody plants that are sparsely used and plant wastes that currently have little or no use. Biomass can provide valuable chemicals and reduce dependence on coal, gas, and fossil fuels, in addition to boosting local and worldwide economies.

In processes separating biomass, several options are available: the OrganoSolv™ and Alcell® processes which are used to efficiently remove the lignin from the other components under mild conditions, kraft pulping, sulfite pulping, pyrolysis, steam explosion, ammonia fiber explosion, dilute acid hydrolysis, alkaline hydrolysis and alkaline oxidative treatment. Kraft pulping is by far the dominant chemical pulping practiced in the world. However, often the removal of lignin from plant biomass can be a costly process, and some research efforts are now aimed at designing plants that either deposit less lignin or produce lignins that are more amenable to chemical degradation in order to avoid separating the biomass components.

Although the cellulosic fraction of biomass has garnered substantial attention recently as a feedstock for ethanol biofuel and other basic chemicals, the intrinsic value of the lignin continues to be largely overlooked. Lignin, which can comprise upwards of 30% of the organic matrix of woody and agricultural biomass, is the most abundant source of aromatic chemicals outside of crude oil. Lignin can be used in developing technologies that transform plant biomass into value-added aromatic chemicals.

Lignin has a complex, polymeric structure whose exact structure is unknown. This large group of aromatic polymers in lignin can be a result from the oxidative combinatorial coupling of 4-hydroxyphenylpropanoids. The aromatic portion of lignin is composed primarily of p-hydroxybenzene, guaiacyl(4-alkyl-2-methoxyphenol), and syringyl(4-alkyl-2,5-dimethoxyphenol) units. The lignin itself may also vary in the ratio of these units depending on its source.

Because of the aromatic portions, lignin can be a source of aromatic chemicals outside of petroleum and coal. Lignin may be obtained from wood and/or agricultural sources. This wood and/or agricultural lignin may be waste lignin from these sources. Lignin can also be obtained from multiple sources that utilize plant material, including pulp and paper mills and the sugar cane milling industries. It is also a major by-product in the biomass-to-ethanol process. Often, these sources of lignin may be considered waste products where there can be an associated cost to dispose of the lignin instead of alternative methods where this lignin can provide value-added materials.

Another source of lignin is the black liquor produced from kraft pulp mills. In the kraft process, black liquor is burnt in a recovery boiler to recover the spent alkali and to generate heat and power for mill operations. Some of the lignin in black liquor could be precipitated and used for value-added applications, especially since a production bottleneck may exist from the thermal capacity of the recovery boiler.

The present invention provides methods for utilizing lignin from biomass, and converting them to value-added biobased materials while minimizing waste products.

### II. SUMMARY OF THE INVENTION

Accordingly, it is an object of the present invention to provide a method for biorefining comprising the steps of providing lignin biomass, processing the lignin biomass, and producing at least one product from said lignin biomass.

According to another embodiment of the invention, the lignin biomass is provided from at least one biomass of plant biomass, woody plant biomass, agricultural plant biomass, and cultivated plant biomass.

According to another embodiment of the invention, the lignin biomass is provided from at least one biomass of fresh biomass, pulp and paper mill biomass, and cellulosic ethanol refinery biomass.

According to yet another embodiment of the invention, the lignin biomass is provided from kraft lignin.

According to still another embodiment of the invention, the lignin biomass is provided from waste lignin.

One object of the present invention is that the waste lignin is provided by at least one waste lignin of cellulosic ethanol refinery waste lignin, bioethanol refinery waste, pulp and paper mill waste, sulfite mill waste lignin, kraft mill waste lignin, and sugar cane milling waste lignin.

Another object of the present invention is the processing of the lignin biomass is provided from at least one process of chemical processing, catalytic processing, biological processing, and pyrolytic processing.

Yet another object of the present invention is providing a lignin pretreatment.

Still another object of the present invention is that the derivative products comprise at least one product from said lignin biomass comprises at least one chemical of biobased aromatic chemicals, biobased aromatic fuels, and lignin residues.

Another object of the present invention is that biobased aromatic chemicals comprise at least one chemical of commodity chemicals, fine chemicals, and specialty chemicals.

Yet another object of the present invention is that at least one product of biobased aromatic chemicals comprise at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils.

Yet another object of the present invention is that at least one product of biobased aromatic chemicals comprise at least chemical of methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, 4-methylguaiacol, 4-methyl-2,6-dimethoxyphenol, guaiacol and 2,6-methoxyphenol, syringol, benzene, toluene, xylene, ethyl benzene, propyl benzene, biphenyl, and pyrolytic oils.

Still yet another object of the present invention is that biobased aromatic fuels comprise at least one chemical of commodity chemicals, fine chemicals, and specialty chemicals.

Still yet another object of the present invention is at least one product of biobased aromatic fuels comprise at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils.

Another object of the present invention is that at least one product of biobased aromatic fuels comprise at least chemical of methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, 4-methylguaiacol, 4-methyl-2,6-dimethoxyphenol, guaiacol and 2,6-methoxyphenol, syringol, benzene, toluene, xylene, ethyl benzene, propyl benzene, biphenyl, and pyrolytic oils.

Still another object of the present invention is the derivative products from the lignin residues comprise at least one chemical of biobased aromatic chemicals and biobased aromatic fuels.

Another object of the present invention is the lignin residues provide energy production.

Yet another object of the present invention is that the energy production provided from the lignin residues is heat or power.

Still another object of the present invention is using at least one product from the lignin biomass in the production of other chemicals, materials, and products.

Still yet another object of the present invention is using at least one of the other chemicals, materials, and products in the production of additional chemicals, materials, and products.

Yet another object of the present invention is that at least one product from the lignin biomass comprises at least one product of achiral, racemic, and optically pure products.

Still yet another object of the present invention is the step of functionalizing the lignin biomass prior to production of at least one product from the lignin biomass.

Still another object of the present invention is that the lignin biomass has a weight, and a waste product of the lignin biomass is less than 20% of the lignin biomass weight.

Another object of the present invention is that the lignin biomass has a weight, and a waste product of the lignin biomass is less than 15% of the lignin biomass weight.

Yet another object of the invention is that it can provide a method of biorefining, comprising the steps of providing lignin biomass comprising at least one biomass of woody biomass, agricultural biomass, kraft biomass, and waste biomass; processing the lignin biomass from at least one process of chemical processing, catalytic processing, biological processing, and pyrolytic processing; functionalizing the lignin biomass prior to producing at least one product from the lignin biomass; producing at least one product from the lignin biomass comprising at least one chemical of biobased aromatic chemicals, biobased aromatic fuels, and lignin residues; producing a plurality of products from the lignin biomass comprising at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils; reducing the waste product of the lignin biomass, wherein the lignin biomass has a weight, and the waste product of said lignin biomass is less than 20% of the lignin biomass weight; and producing energy utilizing the lignin residues.

Further, another object of the present invention can be to provide a method for biorefining that is easy to implement and use.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, and wherein:

FIG. 1 is a flow diagram schematically illustrating the present invention.

FIG. 2 is a flow diagram schematically illustrating another aspect of the present invention.

FIG. 3 is a flow diagram schematically illustrating another aspect of the present invention.

FIG. 4 is a flow diagram schematically illustrating another aspect of the present invention.

FIG. 5 is a flow diagram schematically illustrating another aspect of the present invention.

FIG. 6 is a flow diagram schematically illustrating another aspect of the present invention.

FIG. 7 is a flow diagram schematically illustrating another aspect of the present invention.

FIG. 8 is a flow diagram schematically illustrating another aspect of the present invention.

### IV. DETAILED DESCRIPTION OF THE INVENTION

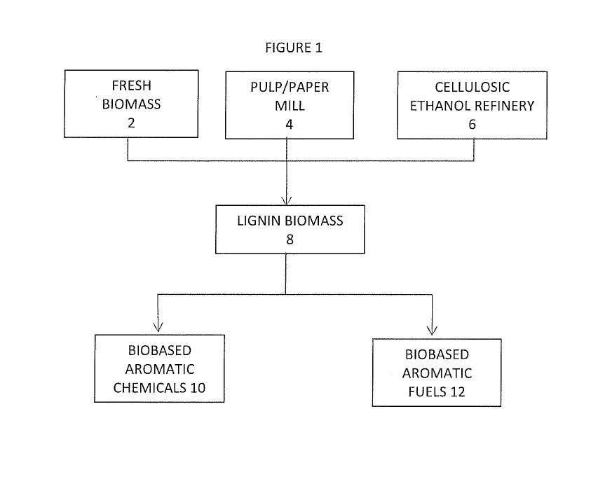

FIG. 1 provides a flowchart overview where lignin biomass 8 from various sources can be converted into other chemical products. The sources for the lignin biomass 8 may include fresh biomass 2, pulp and paper mill 4, and/or cellulose ethanol refinery 6. In processing the lignin biomass 8, it may be converted into other chemical products like biobased aromatic chemicals 10 and biobased aromatic fuels 12.

Lignin may be the most abundant source of aromatic chemicals outside of crude oil. Lignin can be used in developing technologies that transform plant biomass into value-added aromatic chemicals. The sources of lignin biomass 8 may include at least one biomass of plant biomass, woody biomass, agricultural plant biomass, and cultivated plant biomass. The sources of lignin biomass 8 may include fresh biomass 2, pulp and paper mill 4, and/or cellulose ethanol refinery 6. Although these sources of lignin biomass 8 can be used, these sources of lignin biomass 8 are not limited to only those listed herein. No matter the origin of the individual species for the lignin biomass 8, the different sources for lignin biomass 8 may provide a supply for the lignin biomass 8 used within the process described herein.

Lignin within the lignin biomass 8 can be a structurally complex substance made up of p-hydroxybenzene, guaiacyl(4-alkyl-2-methoxyphenol), and syringyl(4-alkyl-2,5-dimethoxyphenol) units. The abundance of each of these units within the lignin biomass 8 may change somewhat between individual species for woody lignin, namely lignin content for hardwoods and softwoods, as well as for agricultural sources and both cultivated and uncultivated plants. This difference in the units based on the species for the lignin biomass 8 can control, or at least predict, the amounts and types of biobased aromatic chemicals 10 and biobased aromatic fuels 12 that may be produced.

To begin the process described herein, fresh biomass 2 may be utilized as a lignin source. Fresh biomass 2 may be considered to be biomass and/or or biomass waste from agricultural, woody, and/or other plant biomass sources. Fresh biomass 2 may also include cultivated plant biomass. Fresh biomass 2 may be used where it may be grown specifically for this application, or where the biomass can be considered a waste product. Some fresh biomass 2 that may be grown specifically for this application may include switchgrass and miscanthus. Some fresh biomass 2 not specifically grown for this application may include agricultural surplus. Applications where the fresh biomass 2 may be used as a waste product include wood chips, corn stover and other agricultural products typically left to rot in fields, and lawn and tree maintenance byproducts. Waste lignin may also be provided from at least one waste lignin of sulfite mill waste lignin, kraft mill waste lignin, and sugar cane mill waste lignin. Where fresh biomass 2 is used, the lignin can be separated from the other components like cellulose, hemicellulose, and other extractives. After the lignin is separated, it may be added to the process described herein.

Pulp and paper mills 4 may also contribute to the lignin biomass 8 from kraft processing. Lignin can be removed during paper processing in a pulp and paper mill 4, where it is typically viewed as an undesirable chemical that requires both energy and chemicals to remove it during pulping. This lignin removal may be done by a chemical removal, with or without mechanical means. Two chemical methods of lignin removal from pulp and paper mills 4 may be kraft processing and sulfite processing.

The most dominant chemical pulping technique employed can be kraft processing, which employs high pHs by using considerable amounts of aqueous sodium hydroxide and sodium sulfide at high temperatures to degrade lignin in a stepwise process. In the kraft process, black liquor can be burnt in a recovery boiler to recover the spent alkali and to generate heat and power for mill operations. However, some of the lignin in black liquor can be precipitated and used for value-added applications where these exist. This conversion to value-added applications may be particularly attractive for a kraft mill where a production bottleneck exists due to the thermal capacity of the recovery boiler. This process may provide kraft lignin.

The sulfite processing yielding lignosulfonates can also be relatively common in the pulp and paper industry. The sulfite process may be conducted between pH 2 and 12 using sulfite with usually either calcium or magnesium as the counterion. The product may be typically soluble in water and in some highly polar organics and amines.

For the cellulose ethanol refineries 6, they may produce lignin biomass 8 and other by-products in the biomass-to-ethanol process, which can also be used to produce the electricity required for the ethanol production process. Cellulosic ethanol refineries produce ethanol fuel. The cellulosic ethanol is made from plant materials like switchgrass and stalks.

Cellulose ethanol refineries 6 may use the Organosolv process or the Alcell process to obtain lignin. Organosolv lignin is obtained by treatment of wood or bagasse, the fibrous residue that remains after plant material may be treated with various organic solvents. The Organosolv process may produce separate streams of cellulose, hemicelluloses, and lignin. It may be considered environmentally friendly because it may not use the sulfides and harsh conditions used in the kraft or lignosulfonate processes, but it does have a higher cost because of the solvent recovery in this process.

Besides the potential sources listed above, another potential source of lignin biomass 8 may include sugar cane milling. Sugar cane milling may provide waste lignin since bagasse, or sugarcane waste fiber, is generated. Bagasse is the name given to the discarded husks of the sugarcane plant after they have been pressed to extract the juices which are refined to make sugar. This agricultural waste is very plentiful and may otherwise be burnt or discarded in the sugar cane milling process. Although several sources for lignin are presented herein, those sources for lignin are not limited to those listed.

Within the process described herein, the lignin biomass 8 may be collected from the various sources which may include but are not limited to fresh biomass 2, pulp and paper mill 4, and/or cellulose ethanol refinery 6, both biobased aromatic chemicals 10 and biobased aromatic fuels 12 may be produced. The production of these chemicals may provide value-added and greener processes in using lignin biomass 8. Producing these chemicals may provide a reduction in the costs associated with waste disposal of lignin and lignin wastes and a means to provide an income in biobased chemical production. In this reduction of waste, the waste product of the lignin biomass may be less than 20% of the lignin biomass weight. It may also be less than 15% of the biomass weight. These waste products, although reduced, may be converted into producing energy which utilizes the waste product.

The processing outlined in FIG. 1 can also address problems with processing lignin 2 without the need to invest large amounts of capital in expensive processing equipment to purify this lignin 2 whether it is a by-product, a waste product, or specifically grown for this application.

The processing outlined in FIG. 1 can further avoid the high costs of converting lignin 2 by conventional means in that the process in FIG. 1 can be low energy and self-contained.

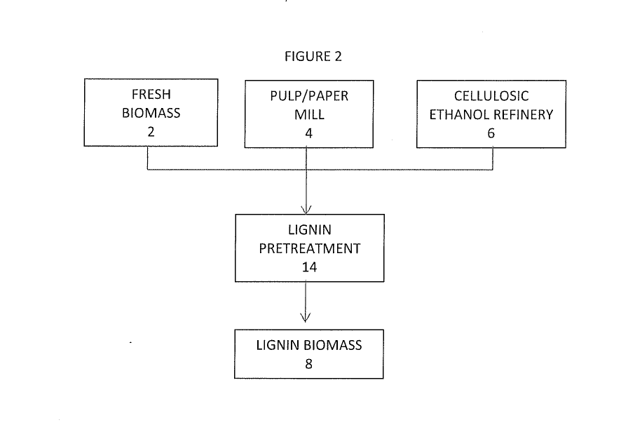

FIG. 2 provides an optional lignin pretreatment 14 within the process. The lignin pretreatment 14 may occur before the lignin biomass 8 can be received from fresh biomass 2, pulp and paper mill 4, and/or cellulose ethanol refinery 6. The purpose of this lignin pretreatment 14 may be to extract additional amounts of lignin biomass 8 from the various sources which may include but are not limited to fresh biomass 2, pulp and paper mill 4, and/or cellulose ethanol refinery 6. This lignin pretreatment 14 may include a series of steps to further separate lignin biomass 8 from the other components of biomass such as cellulose and hemicellulose as well as small amounts of fats, oils, resins, pitches, waxes, and other extractables. A lignin pretreatment process 14 can be described in detail in A METHOD FOR PRODUCING BIOBASED CHEMICALS FROM PLANT BIOMASS (U.S. application Ser. No. 13/292,222 filed Nov. 9, 2011).

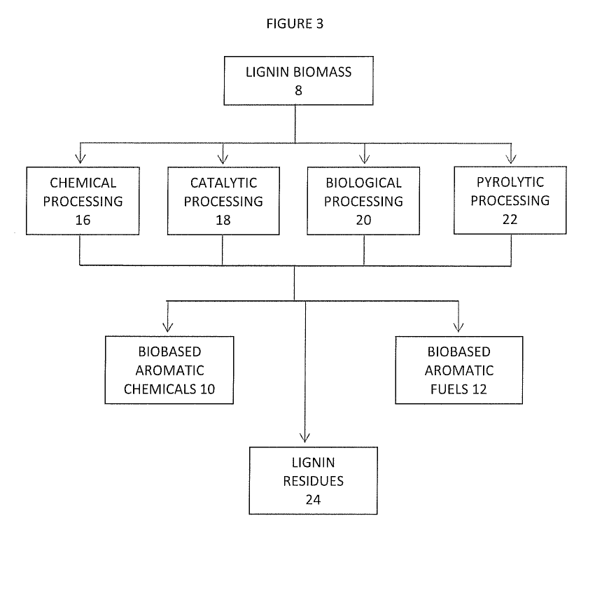

FIG. 3 provides the conversion of lignin biomass 8 into biobased aromatic chemicals 10, biobased aromatic fuels 12, and lignin residues 24. This conversion of the lignin biomass 8 can be through at least one processing step of chemical processing 16, catalytic processing 18, biological processing 20, and pyrolytic processing 22. For the chemical processing 16, catalytic processing 18, biological processing 20, and pyrolytic processing 22, more than one of these steps may be used within the process. Also in the conversion of lignin biomass 8, more than one chemical may be produced, and these chemicals may come from more than one group of biobased aromatic chemicals 10, biobased aromatic fuels 12, and lignin residues 24. For example, the process described herein allows for the production of 4-hydroxybenzaldehyde, vanillin, and syringaldehyde at one time. The ratio of these three products may be controlled by the plant feedstock source of the lignin going into our process. Additionally, the resulting products of biobased aromatic chemicals 10, biobased aromatic fuels 12, and/or lignin residues 24 may be achiral, racemic, and optically pure products. Still, lignin biomass 8 may be functionalized prior to the production of biobased aromatic chemicals 10, biobased aromatic fuels 12, and/or lignin residues 24.

Furthermore, any of the process treatments including but not limited to chemical processing 16, catalytic processing 18, biological processing 20, and/or pyrolytic processing 22 may be conducted under batch or flow conditions for the production of the biobased end-products. There may be more than one of these process treatments used for the production of biobased end-products. Additionally, any of these process treatments may be repeated to provide the requirements for the processing to lignin-based products like biobased aromatic chemicals 10, biobased aromatic fuels 12, and/or lignin residues 24.

For the lignin biomass 8, the chemical processing 16 may also break down the lignin biomass. Some of the chemical processing 16 may include reactions with water at elevated temperatures, base hydrolysis, and oxidation and decarboxylation processes. Many of the chemical processing 16 methods may provide an economic conversion of lignin biomass 8 into other chemicals like biobased aromatic chemicals 10 and biobased aromatic fuels 12, and additional chemicals from the processing of lignin residues 24.

For the lignin biomass 8, the catalytic processing 18 may help to break down the lignin. Lignin comprises a network of ring-like monomeric nine carbon hydrocarbon units that are principally connected by C—O bonds. Cleaving these bonds without breaking open the individual ring structures may produce useful chemical building blocks, rather than a mixture of short chain hydrocarbons. The catalyst used in the catalytic processing 18 may consist of a metal catalyst. In cleaving these bonds, catalysts may contain, but are not limited to, cobalt, nickel, molybdenum, palladium, rhodium, vanadium, or combinations thereof.

For the lignin biomass 8, the biological processing 20 may occur through a number of different methods. These methods for biological processing 20 may use, but are not limited to, enzyme, chemical, and/or microorganism (i.e., bacteria, fungi, etc.) degradation.

For the lignin biomass 8, the pyrolytic processing 22 may also occur. Pyrolytic processing 22 may provide a range of products from the current state of the art: “fast pyrolysis” (or sometimes called “flash pyrolysis”). However, pyrolytic processing 22 may not always be economically attractive.

From the various types of lignin biomass 8 processing, including at least one processing step of chemical processing 16, catalytic processing 18, biological processing 20, and pyrolytic processing 22, biobased aromatic chemicals 10, biobased aromatic fuels 12, and lignin residues 24 may be produced.

Biobased aromatic chemicals 10 may comprise at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils.

Some of the specific biobased aromatic chemicals 10 from lignin can include but are not limited to methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, syringol, benzene, toluene, xylene, ethyl benzene, propyl benzene, biphenyl, and pyrolytic oils.

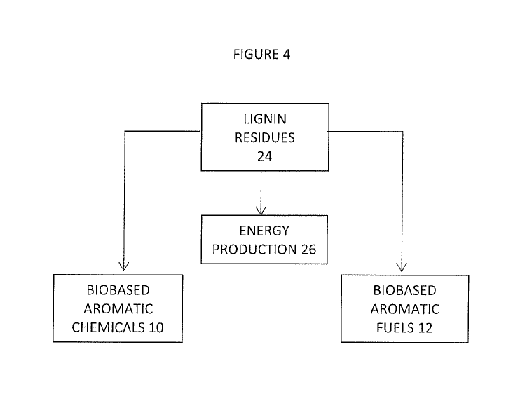

FIG. 4 provides a chart providing depicting the conversion of lignin residues 24 into biobased aromatic chemicals 10, biobased aromatic fuels 12, and/or energy production 26. By converting the lignin residue 24 into useful products, the process described herein may continue to provide additional products and reduce potential waste.

Lignin residue 24 may amount to about 20% of the original lignin biomass 8. Lignin residues 24 may appear to look like lignin. However, lignin residue 24 may be only partially reacted lignin that may be processed specifically to provide either biobased aromatic chemicals 10 and/or biobased aromatic fuels 12, and/or provide energy production 26. Depending on whether biobased aromatic chemicals 10 and/or biobased aromatic fuels 12 may be required, or whether energy production 26 may be needed, the final amounts for these by-products can be tailored to whatever is required for the lignin residue 24. In providing end products or processes for the lignin residues 24, waste in the system can be limited. Lignin residue 24, while considered to typically be a waste product, may provide products to the process which are value-added. These future value-added products of the lignin residue 24 can provide a biobased source of these chemical product(s) that may be typically derived from petroleum.

First, lignin residues 24 may be used to produce biobased aromatic chemicals 10. The production of these biobased aromatic chemicals 10 may be completed through at least one processing step of chemical processing 16, catalytic processing 18, biological processing 20, and pyrolytic processing 22, as described in FIG. 3. These biobased aromatic chemicals 10 may include at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils. Specifically, these biobased aromatic chemicals 10 can include but are not limited to methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, syringol, benzene, toluene, xylene, ethyl benzene, propyl benzene, biphenyl, and pyrolytic oils.

Second, lignin residues 24 may also be used to produce biobased aromatic fuels 12. The production of these biobased aromatic fuels 12 may be completed through at least one processing step of chemical processing 16, catalytic processing 18, biological processing 20, and pyrolytic processing 22, as described in FIG. 3.

For the energy production 26, several methods may be used to provide energy to the production facility from the lignin residues 24. This energy production 26 may be in the form of heat, steam, and/or power. These methods for energy production 26 may also be used to provide energy directly to the production facility, or to other places besides the production facility in which the energy is generated. The methods for energy production 26 may include but are not limited to combustion, gasification, and/or pyrolysis.

Lignin and lignin residue combustion may be a current method used in paper mills to produce process heat, power, and/or steam to recover pulping chemicals. Lignin combustion may be beneficial in that it may be considered to provide zero CO₂ emissions.

Lignin gasification may produce syngas (also known as “Synthesis gas”), which is a gas mixture comprised of a combination of carbon monoxide and hydrogen gases. In this gasification process within the biorefinery, the lignin-rich residue from lignocellulosics are fed into the gasifier, where the sugars are primarily converted into ethanol while the lignin is primarily converted into syngas products. While syngas may only have 50% of the energy density of natural gas, it can be burnt and is used as a fuel source.

Lignin pyrolysis may also provide energy production 26. Additionally, lignin can also be converted into reformulated gasolines in hydroliquefication. Although still in its infancy, hydroliquefication may also be a reasonable form of energy production 26 in the future.

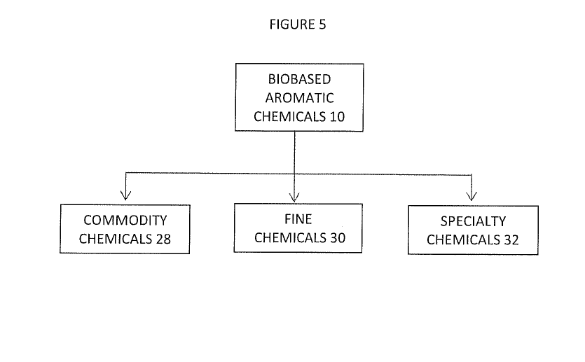

FIG. 5 provides a diagram depicting the conversion of biobased aromatic chemicals 10 formed from the various reactions in FIG. 3 to commodity chemicals 28, fine chemicals 30, and/or specialty chemicals 32. The biobased aromatic chemicals 10 may provide chemicals which may be produced based on what may be required and/or how the lignin content, composition, quality, and/or distribution of the lignin biomass 8 may be used.

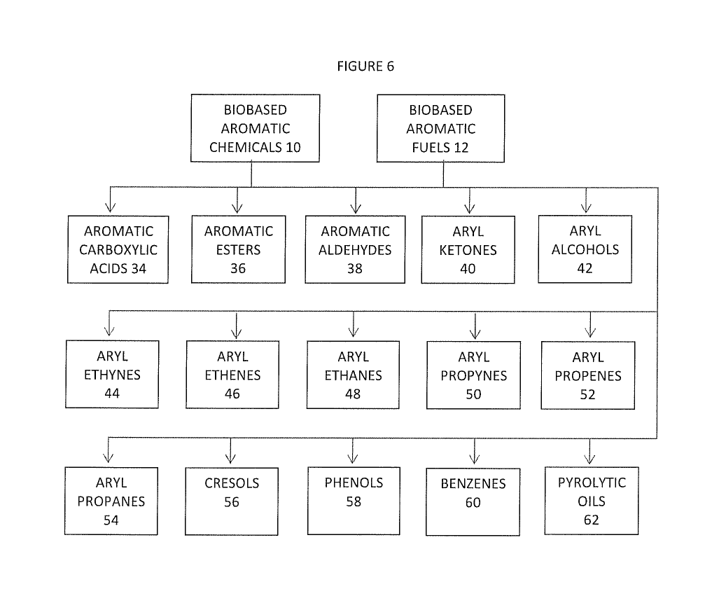

FIG. 6 provides a diagram depicting the conversion of biobased aromatic chemicals 10 and/or biobased aromatic fuels 12 into a variety of products. These products may include aromatic carboxylic acids 34, aromatic esters 36, aromatic aldehydes 38, aryl ketones 40, aryl alcohols 42, aryl ethynes 44, aryl ethenes 46, aryl ethanes 48, aryl propynes 50, aryl propenes 52, aryl propanes 54, cresols 56, phenols 58, benzenes 60, and/or pyrolytic oils 62. The biobased aromatic chemicals 10 and/or the biobased aromatic fuels 12 may provide chemicals which may be produced based on what may be required and/or how the lignin content, composition, quality, and/or distribution of the lignin biomass 8 may be used.

The diagram in FIG. 6 provides some of the chemicals that may be derivative products of biobased aromatic chemicals 10 and/or biobased aromatic fuels 12. Although the derivative products listed provide many of the potential derivative products, the derivative products are not limited to those listed in this figure. The derivative products may be commodity chemicals, fine chemicals, and specialty chemicals. Additionally, they may be achiral, racemic, and optically pure products. These derivative products may be produced through a chemical process, biological process, catalytic process, and/or pyrolytic process.

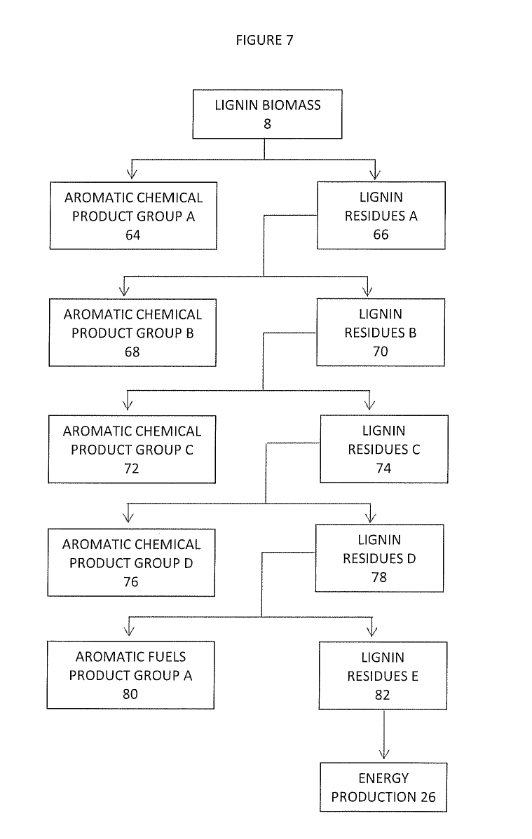

FIG. 7 provides a diagram depicting the conversion of lignin biomass 8 to several different products, including a tiered production of different chemical products from the lignin residues. From FIG. 7, the lignin biomass 8 may be converted into an aromatic chemical product group A 64 or into lignin residues A 66. Subsequently, the lignin residues A 66, may then be converted into an aromatic chemical product group B 68 and/or into lignin residues B 70. Next, the lignin residues B 70 may then be converted into an aromatic chemical product group C 72 and/or into lignin residues C 74. From there, the lignin residues C 74, may then be converted into an aromatic chemical product group D 76 and/or into lignin residues D 78. After that the lignin residues D 78, may then be converted into an aromatic fuels product group A 80 and/or into lignin residues E 82. The lignin residues E 82 may also be converted into energy production 26.

Within the process described in FIG. 7, any of the steps described may be omitted and/or modified. Additional tiers within the tiered chemical production process may also be added. From FIG. 7, a tiered chemical processing may occur where a plurality of different chemicals may be produced from the lignin biomass 8.

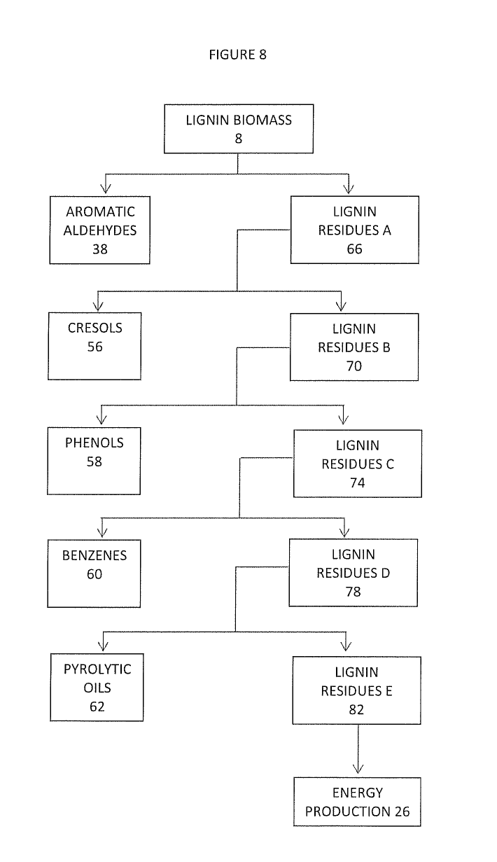

FIG. 8 shows an example of what the aromatic chemical product groups shown in FIG. 7 may be. Here, the lignin biomass 8 may be converted into aromatic aldehydes 38 or into lignin residues A 66. Subsequently, the lignin residues A 66, may then be converted into cresols 56 and/or into lignin residues B 70. Next, the lignin residues B 70 may then be converted into phenols 58 and/or into lignin residues C 74. From there, the lignin residues C 74, may then be converted into benzenes 60 and/or into lignin residues D 78. After that the lignin residues D 78, may then be converted into pyrolytic oils 62 and/or into lignin residues E 82. The lignin residues E 82 may also be converted into energy production 26.

The embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

I/We claim:

1. A method for biorefining, comprising the steps of:
providing lignin biomass;
processing said lignin biomass; and
producing at least one product from said lignin biomass.

2. The method of claim 1, wherein said lignin biomass is provided from at least one biomass of plant biomass, woody plant biomass, agricultural plant biomass, and cultivated plant biomass.

3. The method of claim 1, wherein said lignin biomass is provided from at least one biomass of fresh biomass, pulp and paper mill biomass, and cellulosic ethanol refinery biomass.

4. The method of claim 1, wherein said lignin biomass is provided from kraft lignin.

5. The method of claim 1, wherein said lignin biomass is provided from waste lignin.

6. The method of claim 5, wherein said waste lignin is provided from at least one waste lignin of sulfite mill waste lignin, kraft mill waste lignin, and sugar cane mill waste lignin.

7. The method of claim 1, wherein said processing of said lignin biomass is provided from at least one process of chemical processing, catalytic processing, biological processing, and pyrolytic processing.

8. The method of claim 7, further comprising the step of providing a lignin pretreatment.

9. The method of claim 1, wherein said at least one product from said lignin biomass comprises at least one chemical of biobased aromatic chemicals, biobased aromatic fuels, and lignin residues.

10. The method of claim 9, wherein said biobased aromatic chemicals comprise at least one chemical of commodity chemicals, fine chemicals, and specialty chemicals.

11. The method of claim 10, wherein said at least one product of biobased aromatic chemicals comprise at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils.

12. The method of claim 11, where said comprises at least one product of biobased aromatic chemicals comprise at least chemical of methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, 4-methylguaiacol, 4-methyl-2,6-dimethoxyphenol, guaiacol and 2,6-methoxyphenol, syringol, benzene, toluene, xylene, ethyl benzene, propyl benzene, biphenyl, and pyrolytic oils.

13. The method of claim 9, wherein said biobased aromatic fuels comprise at least one chemical of commodity chemicals, fine chemicals, and specialty chemicals.

14. The method of claim 13, wherein said at least one product of biobased aromatic fuels comprise at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils.

15. The method of claim 14, where said comprises at least one product of biobased aromatic chemicals comprise at least chemical of methyl and ethyl 4-hydroxybenzoate, methyl and ethyl vanillate, methyl and ethyl syringate, 4-hydroxybenzoic acid, (4-hydroxyphenyl)acetic acid, vanillic acid, homovanillic acid, syringic acid, homosyringic acid, 4-hydroxybenzaldehyde, vanillin, syringaldehyde, 4-hydroxybenzyl alcohol, 2-(4-hydroxyphenyl)ethanol, vanillyl alcohol, homovanillyl alcohol, syringyl alcohol, homosyringyl alcohol, 4-hydroxyacetophenone, acetoguaiacone, acetosyringone, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, iso-syringeugenol, ethyl phenol, ethyl guaiacol, ethyl syringol, propyl phenol, propyl guaiacol, propyl syringol, cresol, creosol, syringyl creosol, phenol, guaiacol, 4-methylguaiacol, 4-methyl-2,6-dimethoxyphenol, guaiacol and 2,6-methoxyphenol, syringol, benzene, toluene, xylene, ethyl benzene, propyl benzene, biphenyl, and pyrolytic oils.

16. The method of claim 9 wherein said lignin residues comprise at least one chemical of biobased aromatic chemicals and biobased aromatic fuels.

17. The method of claim 9, wherein said lignin residues provide energy production.

18. The method of claim 17, wherein said energy production is heat or power.

19. The method of claim 1, further comprising the step of:
using at least one product from said lignin biomass in the production of other chemicals, materials, and products.

20. The method of claim 19, further comprising the step of:
using at least one of said other chemicals, materials, and products in the production of additional chemicals, materials, and products.

21. The method of claim 1, wherein at least one product from said lignin biomass comprises at least one product of achiral, racemic, and optically pure products.

22. The method of claim 1, further comprising the step of functionalizing said lignin biomass prior to said producing at least one product from said lignin biomass.

23. The method of claim 1, wherein said lignin biomass has a weight, and a waste product of said lignin biomass is less than 20% of said lignin biomass weight.

24. The method of claim 1, wherein said lignin biomass has a weight, and a waste product of said lignin biomass is less than 15% of said lignin biomass weight.

25. A method for biorefining, comprising the steps of:
providing lignin biomass comprising at least one biomass of woody biomass, agricultural biomass, kraft biomass, and waste biomass;
processing said lignin biomass from at least one process of chemical processing, catalytic processing, biological processing, and pyrolytic processing;
functionalizing said lignin biomass prior to producing at least one product from said lignin biomass;
producing at least one product from said lignin biomass comprising at least one chemical of biobased aromatic chemicals, biobased aromatic fuels, and lignin residues;
producing a plurality of products from said lignin biomass comprising at least one chemical of aromatic carboxylic acids, aromatic esters, aromatic aldehydes, aryl ketones, aryl alcohols, aryl ethanes, aryl ethenes, aryl ethynes, aryl propanes, aryl propenes, aryl propynes, cresols, phenols, benzenes, and pyrolytic oils;
reducing the waste product of said lignin biomass, wherein said lignin biomass has a weight, and said waste product of said lignin biomass is less than 20% of said lignin biomass weight; and
producing energy utilizing said lignin residues.
